(19) United States
(12) Patent Application Publication
(10) Pub. No.: US 2025/0191771 A1
(43) Pub. Date: Jun. 12, 2025

(54) DETERMINATION OF CARDIAC FUNCTIONAL INDICES

(71) Applicant: University of Leeds, Leeds (GB)

(72) Inventors: Alejandro F. Frangi, Leeds (GB); Andres Diaz-Pinto, Leeds (GB); Nishant Ravikumar, Leeds (GB)

(21) Appl. No.: 18/856,298

(22) PCT Filed: Apr. 11, 2022
(86) PCT No.: PCT/IB2022/053356

§ 371 Date: Oct. 11, 2024

(51) Int. Cl. G16H 50/30; A61B 3/12; A61B 3/14; A61B 5/00; G06T 7/00

(52) U.S. Cl. CPC G16H 50/30; A61B 3/12; A61B 3/14; A61B 5/7275; G06T 7/0012; G06T 2207/20084; G06T 2207/30041; G06T 2207/30048

(57) ABSTRACT

A computer-implemented method for determining cardiac functional indices for a patient including: receiving an image of a fundus of the patient; encoding the received image into a joint latent space; decoding from the joint latent space a representation of the patient's heart; providing the representation decoded from the joint latent space to a neural network configured to generate cardiac functional indices; and outputting the cardiac functional indices generated by the neural network in response to receiving the decoded representation of the patient's heart.

### FIELD

The present invention relates to a computer-implemented method for estimating cardiac functional indices for a patient.

### BACKGROUND

Cardiovascular diseases (CVD) represent a major cause of death and socio-economic burden globally. According to the World Health Organization, there are an estimated 17.9 million CVD-related deaths worldwide annually. Identification and timely treatment of CVD risk factors is a key strategy for reducing CVD prevalence in populations and for risk modulation in individuals.

Conventionally, CVD risk is determined using demographic and clinical parameters such as age, sex, ethnicity, smoking status, family history and a history of hyperlipidaemia, diabetes mellitus or hypertension. Imaging tests such as coronary CT imaging, echocardiography, and cardiovascular magnetic resonance (CMR) help stratify patient risk by assessing coronary calcium burden, myocardial scar burden, ischemia, cardiac chamber size and function. Cardiovascular imaging, however, is usually only performed in secondary care and is relatively expensive, limiting its availability in less-developed and developing countries. In developed countries, in turn, access prioritization to advance cardiovascular imaging in high-risk patients may avoid collapsing healthcare services and cost-effective use of resources.

### SUMMARY

It is an object of the present invention to obviate or mitigate one or more of the problems set out above.

In an example described herein there is a computer-implemented method (100) for determining cardiac functional indices (111) for a patient comprising: receiving an image of a fundus of the patient (101); encoding the received image (101) into a joint latent space, decoding from the joint latent space a representation of the patient's heart (106), providing the representation decoded from the joint latent space to a neural network configured to generate cardiac functional indices (110); and outputting the cardiac functional indices generated by the neural network in response to receiving the decoded representation of the patient's heart. Advantageously the method may allow cardiac functional indices of a patient to be determined by using a captured fundus image rather than a captured image of the heart (such as a CMR image or a CT image).

The representation decoded from the joint latent space may be, for example, an image of the patient's heart, such as a CMR image or CT image. Alternatively, the representation decoded from the joint latent space may be an abstract representation of the patient's heart. The abstract representation may be a lower-dimensional abstract representation (compared to a decoded image).

The method may further comprise use of a neural network (109) configured to process an input and provide an output. The input to the neural network (109) may be a first characteristic of the patient (108). The output from the neural network (109) may be provided to the neural network configured to determine cardiac functional indices (110). Beneficially, including patient characteristics as input data may provide the method with increased sensitivity for determining cardiac functional indices. The first characteristic may be one of a plurality of first characteristics.

The method may further comprise use of a neural network configured to process the representation decoded from the joint latent space (107). The input to the neural network configured to process the representation decoded from the joint latent space (107) may comprise the representation decoded from the joint latent space (106). The output of the neural network configured to process the representation decoded from the joint latent space (107) may be provided as an input for the neural network configured to determine cardiac functional indices (110).

The neural network configured to process the representation decoded from the joint latent space (107) may be a convolutional neural network (CNN).

The determined cardiac functional indices (111) may comprise the left ventricular mass (LVM), the left ventricular end-diastolic volume (LVEDV), ejection fraction (EF), cardiac output (CO), LV end-systolic volume (LVESV), regional wall thickening (WT), regional wall motion (WM) and/or myocardial strains among others.

The method may further determine the patient's risk of adverse cardiovascular characteristics or events (114). The method may further comprise a neural network for determining the patient's risk of adverse cardiovascular characteristics/events (112). The input to the neural network for predicting the patient's risk of adverse cardiovascular characteristics/events (112) may comprise the determined cardiac functional indices (111). Beneficially, this may provide a method for predicting a patient's risk of adverse cardiovascular characteristics/events without the need for imaging of the patient's heart, and which provides greater accuracy than prior art methods.

The input to the neural network for determining the patient's risk of adverse cardiovascular characteristics/events (112) may further comprise second patient characteristics (113). The second patient characteristics (113) may comprise the same or different characteristics to the first patient characteristics (108). Advantageously, this may increase the method's accuracy in determining the patient's risk of adverse cardiovascular characteristics/events.

Computer-readable media may comprise instructions which, when executed by one or more computers, cause the one or more computers to perform any one or more of the methods disclosed herein. One or more computers may be configured to carry out the method disclosed herein. In some example systems, the one or more computers may comprise one or more wearable devices, such as digital retinal implants or bio-sensing glasses.

### LIST OF FIGURES

Embodiments of the invention will now be described, by way of example only, with reference to the accompanying schematic drawings, in which:

FIG. 1 is a schematic depiction of an example of a system for determining cardiac functional indices and/or predicting risk of adverse cardiovascular events;

FIG. 2 is a flowchart depicting an example of a method of predicting the risk of adverse cardiovascular events;

FIG. 3 is a flowchart depicting an example method of training the system of FIG. 1 to determine cardiac functional indices and/or predict the risk of adverse cardiovascular events;

FIG. 4 is a schematic depiction of an example method for training the multichannel variational auto-encoder (mcVAE) depicted in FIG. 1;

FIG. 5a shows plots of the performance of the system of FIG. 1 when determining cardiac functional indices compared to manual annotations on CMR images;

FIG. 5b shows plots of the performance of the system of FIG. 1 for determining cardiac functional indices compared to automatic annotations o CMR images;

FIG. 6 shows plots comparing the performance of the system of FIG. 1 for estimating cardiac functional indices with different resolutions of captured fundus image;

FIG. 7 shows plots the performance of the system of FIG. 1 for predicting the risk of adverse cardiovascular events;

FIG. 8 compares the performance of the system of FIG. 1 for predicting risk of adverse cardiovascular events when using different patient characteristics;

FIG. 9 shows example distributions of latent variables after encoding using the joint latent space depicted FIG. 1;

FIG. 10 shows an example distribution of Frechet Inception Distance scores for representations of the patient's heart decoded from the joint latent space depicted in FIG. 1;

FIG. 11 shows example values for the regressor coefficients in the method of FIG. 2; and

FIG. 12 depicts an example computer system that may be used to perform the methods described herein.

### DETAILED DESCRIPTION

The apparatus and methods as disclosed herein may make use of retinal images (e.g., fundus photography or optical coherence tomography scans, optionally together with patient characteristics and/or demographic data) to determine (or predict/estimate) one or more cardiac functional indices by jointly learning a latent space of retinal and CMR images. Cardiac functional indices may include, for example, the left ventricle (LV) mass (LVM), the LV end-diastolic volume (LVEDV), ejection fraction (EF), cardiac output (CO), LV end-systolic volume (LVESV), regional wall thickening (WT), regional wall motion (WM) and myocardial strains among others. A review of at least some of the relevant indices is provided in Frangi A F, Niessen W J, Viergever M A. Three-dimensional modeling for functional analysis of cardiac images: a review. IEEE Trans Med Imaging. 2001 January; 20 (1): 2-25.

Cardiac functional indices (optionally together with patient characteristics and/or demographic data) may be used to determine (or predict/estimate) a risk of adverse cardiovascular characteristics/events. Such adverse cardiovascular characteristics may include, for example, arrhythmias, heart valve disease, cardiomyopathy (enlarged heart), carotid or coronary artery disease, among others, each of which may result in adverse events such as myocardial infarction.

By use of apparatuses and methods described herein to determine cardiac functional indices, patients may be assessed for risk of adverse cardiovascular events at routine ophthalmic or optician visits using readily available equipment, rather than needing to attend a specialist cardiologist or requiring specialist equipment such as a magnetic resonance imaging (MRI) or a computed tomography (CT) scanner. Assessment for risk of adverse cardiovascular events at routine ophthalmic visits may further enable timely referral of the patient for further examination. Additionally, or alternatively, cardiac functional indices may also be used to identify, diagnose and/or monitor signs, incidents or indicators of possible pathological cardiac remodelling and/or hypertension. By recording or monitoring a patient's cardiac functional indices over time, problematic changes may be observed more quickly and easily, enabling the patient to be referred for further assessment to cardiologists (for example, in the event of detection of a significant change in the cardiac functional indices). An ophthalmologist, an optician, or an automated risk detection system may record a patient's cardiac functional indices over time and/or determine the risks of adverse events. The monitoring of a patient may also be performed during clinical trials to observe responses to interventions, which may reduce the cost and complexity of clinical trials. Further, the use of more readily available equipment may allow clinical trial participants to be more geographically dispersed, which may enable the inclusion of representative cohorts within the trial.

Cardiac functional indices or risk of adverse cardiovascular events may be used for additional purposes. Examples of other purposes include a more personalized calculation (compared to a calculation not using cardiac functional indices or risk of adverse cardiovascular events) of health insurance premiums and automatic generation and output of dietary and lifestyle adaptations. Further intelligent personalization could be achieved by integration in retinal implants or specialized head-mounted devices.

The system may be implemented on a computer system, which may comprise one or more computers. For example, the computer system may comprise or be part of a wearable device configured to capture retinal images. The system may be used for screening patients at routine opticians check-ups or used as an indicator for secondary referrals in eye clinics. All patients (e.g. in an optician or eye clinic) may be screened, or alternatively, only high-risk patients (i.e. those identified, based on one or more patient's characteristics, as having a high risk of suffering adverse cardiovascular events) may be screened. The computer system may comprise retinal implants and/or a head-mounted component, for example, smart glasses or a AR/VR headset.

For example, smart glasses (i.e. a pair of smart glasses) may comprise a camera. For example, smart glasses may comprise a camera for eye-tracking. The camera of the smart glasses may be configured, in accordance with the present techniques, to record images of one or both eyes of an user while the user is wearing the smart glasses. The images may be still images or the images may be moving images (i.e. a digital film) from which one or more still images may be extracted. The still images may images a portion of the fundus of an eye of the wearer. The still images may then be analysed by the smart glasses to determine cardiac functional indices and/or to determine risk of adverse cardiovascular characteristics or events. Additionally or alternatively, the still images (or the moving images from which one or more still images may be extracted) may be transmitted (e.g. wirelessly, for example, over a WLAN or 4G network, or wired) to a computer (e.g. a server) and the server may analyse (and, optionally, extract) the still images to determine cardiac functional indices and/or to determine risk of adverse cardiovascular characteristics or events.

Output from the system (i.e. the determined cardiac functional indices and/or the determined risk of adverse cardiovascular characteristics or events) may be provided to the user, for example, through a user (I/O) interface of the smart glasses (e.g. on a heads-up display) or a computer of the computer system (e.g. on the display of a smart phone deviced paired with the smart glasses). The output may comprise a number incidating a value of the determined cardiac functional indices and/or the determined risk of adverse cardiovascular characteristics or events. Additionally or alternatively, the output may prompt the user to, for example, consult a medical professional. As a further addition or alternative, the output may be provided to, or cause a communication (for example, an email to be sent by the computer) with a medical professional prompting the medical professional to contact the user.

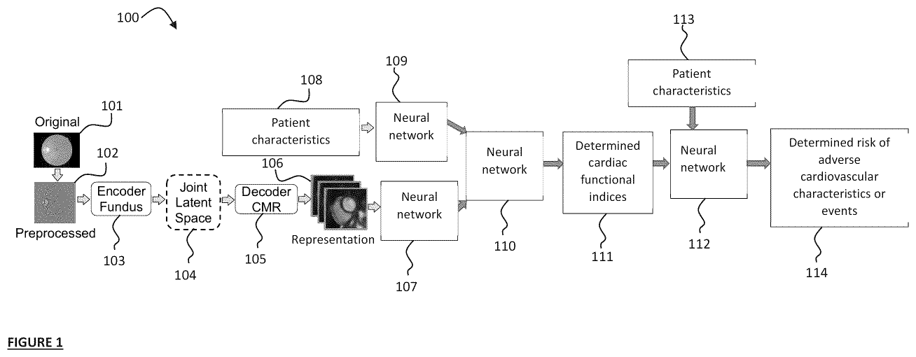

FIG. 1 depicts an example of a computer-implemented system 100 for predicting cardiac functional indices based on retinal images.

As depicted in FIG. 1, a captured fundus image 101 (i.e., an image of a patient's fundus captured by an image sensor) is received. A captured fundus image 101 may be received from a camera or any other appropriate image sensor as will be readily apparent to the skilled person. For example, the camera may comprise a camera of a portable user device such as a smartphone. At a first step, the captured fundus image 101 may be preprocessed to generate a preprocessed fundus image 102. Examples of preprocessing operations that may be performed are described in more detail below. In general, however, it will be understood that preprocessing is not an essential feature of the techniques described herein. For example, the captured fundus image 101 may be received in a format that does not require preprocessing. Therefore, the following references to the further operations performed on the captured fundus image 101 are to be understood to be operations performed on the captured fundus image 101 or the pre-processed captured fundus image 102 as appropriate.

The captured fundus image 101 is encoded into a latent space 104, for example, by use of a multi-channel variational autoencoder (mcVAE). The latent space 104 is a joint latent space providing an embedding of both fundus images and cardiac magnetic resonance (CMR) images, as described below. The joint latent space 104 may be obtained by other techniques, for example, a Bayesian mixture of expert models or disentangled representation learning techniques.

As will be known to the skilled person, a cardiac magnetic resonance (CMR) image may be one or more 3D images or one or more 2D images. A plurality of 3D or 2D images may provide a temporal sequence of (2D or 3D) images (e.g. across one or more complete cardiac cycles). A plurality of 2D images may correspond to a trajectory of 2D slices of 3D space. It will also be apparent to the skilled person that while CMR images are discussed in the following examples, other imaging techniques may be used to obtain images of the patient's heart, such as CT scans.

A representation of the patient's heart 106 may be decoded from the joint latent space 104 using a decoder (or generative model) 105. The representation may be, for example, a representation of a CMR image (or another type of image used to train the joint latent space 104). Alternatively, the representation may be an abstract representation of the patient's heart (e.g., an output may be obtained from the joint latent space 104 without generating a full image representation, but which nonetheless represents the patient's heart). For the purpose of example below, it is assumed that a CMR image is decoded from the joint latent space 104. An example decoder architecture is shown in Table 1 below, which is particularly effective, although it will be appreciated that other decoder architectures may be used. In Table 1, each row corresponds to a layer of the respective model. The representation of the patient's heart 106 is provided as an input to a neural network 107 configured to process the representation of the patient's heart 106. An output from the neural network 107 may be a lower-dimensional representation of the representation of the patient's heart 106. Patient characteristics 108 (of the patient from which the fundus image 101 was obtained) are provided as an input to a neural network 109. An output from the neural network 109 may be a lower-dimensional representation of the patient characteristics 108.

The outputs from the neural network 107 and neural network 109 (i.e. the neural network 107 configured to process the representation of the patient's heart 106 and the neural network configured to process the patient characteristics 108) are provided as one or more inputs to a neural network 110 configured to determine cardiac functional indices. The outputs from the neural network 107 and neural network 109 may be provided as separate inputs (i.e. two separate inputs) to the neural network 110 configured to determine cardiac functional indices or, alternatively, concatenated (or otherwise fused) together prior to being provided as a single input to the neural network 110 configured to determine cardiac functional indices. The neural network 110 configured to determine cardiac functional indices outputs determines cardiac functional indices 111. The determined cardiac functional indices 111, optionally together with patient characteristics 113, may be provided as an input to a neural network 112 configured to predict the patient's risk of adverse cardiovascular events. The neural network 112 configured to predict the patient's risk of adverse cardiovascular events outputs indicated risks of adverse cardiovascular events 114.

TABLE 1
Example encoder and decoder architectures. In the example retinal
encoder architecture, the third dimension in the input layer
corresponds to the three channels of each pixel in the image
and correspond to red, green and blue channels.

| Channel | Encoder | Decoder |
|---|---|---|
| Retinal | Input 128 × 128 × 3 | Latent variables, [symbol] C, Reshape |
| | 3 × 3 conv, | 3 × 3 ConvTranspose2d, |
| | LeakyReLU (alpha 0.2), | ReLU, Dropout 0.15 |
| | Dropout 0.15 | |
| | 3 × 3 conv, | 3 × 3 ConvTranspose2d, |
| | LeakyReLU (alpha 0.2), | ReLU, Dropout 0.15 |
| | Dropout 0.15 | |
| | 3 × 3 conv, | 3 × 3 ConvTranspose2d, |
| | LeakyReLU (alpha 0.2), | ReLU, Dropout 0.15 |
| | Dropout 0.15 | |
| | 3 × 3 conv, | 3 × 3 ConvTranspose2d, |
| | LeakyReLU (alpha 0.2), | ReLU, Dropout 0.15 |
| | Dropout 0.15 | |
| | 3 × 3 conv, | 3 × 3 ConvTranspose2d, |
| | LeakyReLU (alpha 0.2), | ReLU, Dropout 0.15 |
| | Dropout 0.15 | |
| | [symbol] C output layer, | T [symbol] activation, |
| | Latent variables | Reconstructed Retinal image |
| CMR | Input 128 × 128 × 35 | Latent variables, [symbol] C, Reshape |
| | 3 × 3 conv, | 3 × 3 ConvTranspose2d, |
| | LeakyReLU (alpha 0.2), | ReLU, Dropout 0.15 |
| | Dropout 0.15 | |
| | 3 × 3 conv, | 3 × 3 ConvTranspose2d, |
| | LeakyReLU (alpha 0.2), | ReLU, Dropout 0.15 |
| | Dropout 0.15 | |
| | 3 × 3 conv, | 3 × 3 ConvTranspose2d, |
| | LeakyReLU (alpha 0.2), | ReLU, Dropout 0.15 |
| | Dropout 0.15 | |
| | 3 × 3 conv, | 3 × 3 ConvTranspose2d, |
| | LeakyReLU (alpha 0.2), | ReLU, Dropout 0.15 |
| | Dropout 0.15 | |
| | 3 × 3 conv, | 3 × 3 ConvTranspose2d, |
| | LeakyReLU (alpha 0.2), | ReLU, Dropout 0.15 |
| | Dropout 0.15 | |
| | [symbol] C output layer, | T [symbol] activation, |
| | Latent variables | Reconstructed CMR image |

[symbol]

indicates data missing or illegible when filed

The captured fundus image 101 may have been captured previously and stored in computer-readable memory. The captured fundus image 101 may be received by the system of FIG. 1 from another entity over a network, such as the Internet. Alternatively, the captured fundus image 101 may be used as an input for the system of FIG. 1 contemporaneously with its capture. The captured fundus image 101 may be a digital image captured using a digital camera. Alternatively, the captured fundus image 101 may be a digital image formed by scanning an analogue film or an analogue photographic image. As a further alternative, the captured fundus image 101 may be formed using any direct fundus scanning methodology that uses any physical or physiological contrast from the eye health condition.

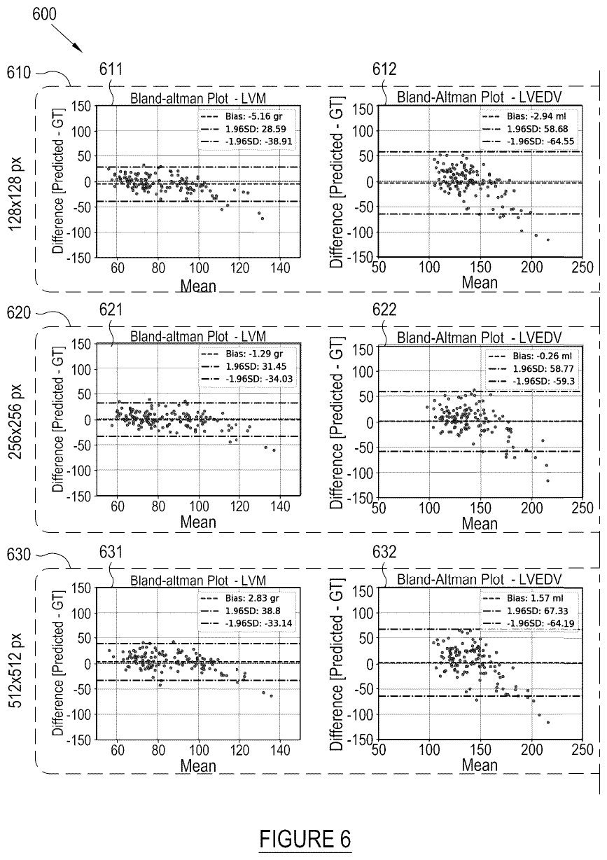

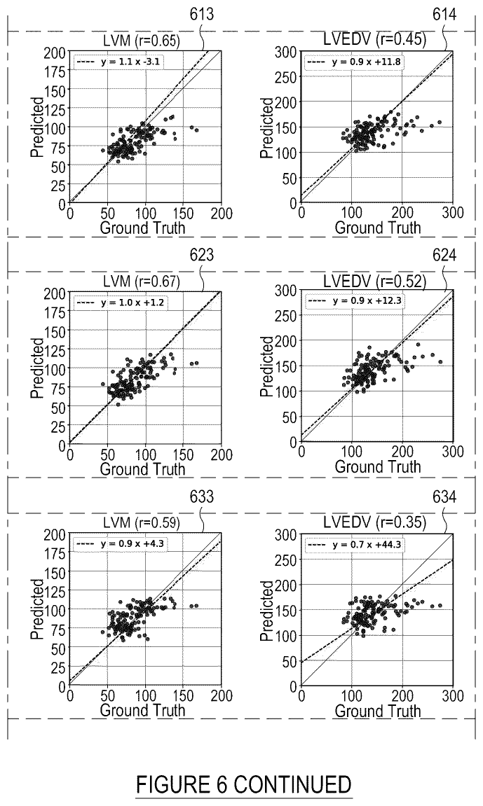

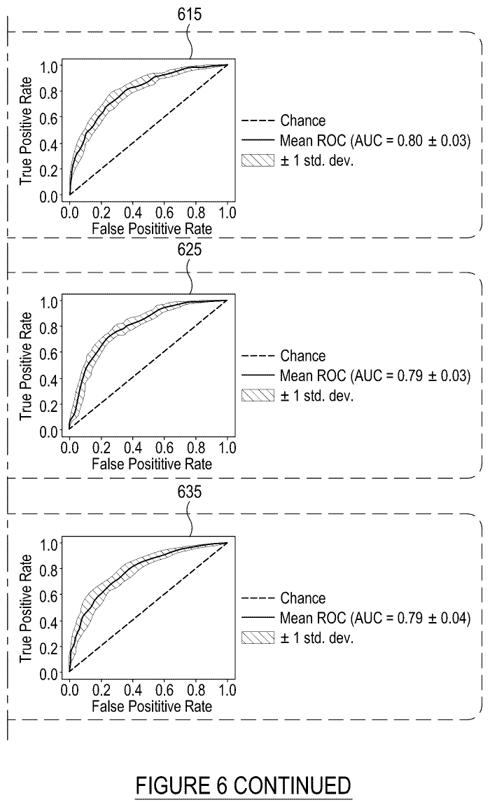

As described above, preprocessing may be applied to the captured fundus image 101. Preprocessing may comprise one or more preprocessing steps. For example, the captured fundus image 101 may be cropped, filtered, enhanced, restored, interpolated, super-resolved, or otherwise. For example, if the captured fundus image 101 contains unnecessary (or uninformative) information (e.g., pixels that do not depict the patent's fundus), the captured fundus image 101 may be cropped to remove the unnecessary information. The radius of the field-of-view may be determined using appropriate thresholding techniques to identify, for example, foreground pixels. Such thresholding techniques are well known to the person skilled in the art and are not described in detail herein. Another example of preprocessing that may be performed is that the captured fundus image 101 may be resampled from a first image resolution (for example, 2048×1536 pixels) to a second image resolution. The first image resolution may depend on the image sensor used to capture the captured fundus image 101. The second image resolution preferably may be 128×128 pixels, or alternatively, 256×256 pixels, 512×512 pixels or otherwise. A second image resolution of 128×128 pixels may be preferable as experimental data, shown in FIG. 6 and discussed below, suggests that such an image resolution produces better discrimination than other image resolutions. The image resolution and resampling may alternatively be referred to as the image size and resizing, respectively. Resampling the captured fundus image 101 may reduce computational resources required for subsequent processing or optimally fit embedded hardware constraints. As another example of preprocessing that may be performed, a process referred to as “unsharp masking” may be applied to the captured fundus image 101. Unsharp masking subtracts a smoothed image from an original image (which may be the captured fundus images with or without other preprocessing applied). For example, a smoothed image may be produced by applying a Gaussian filter (e.g. of size 4×4 pixels) on the captured fundus image and the smoothed image then subtracted from the captured fundus image. Any one or more of the above-described preprocessing steps may be applied to the captured fundus image 101. Alternatively or additionally, other applicable preprocessing steps may be used as will be known to the skilled person.

A mcVAE uses a multivariate technique that jointly analyses heterogeneous data by projecting observations from different sources into a joint latent space (also referred to as a common latent space, hidden space or an embedding space). As discussed above, the captured fundus image 101 may be encoded into the joint latent space 104 using a mcVAE comprising one or more encoders 103 and one or more decoders 105. mcVAEs may be considered extensions to variational auto-encoders (VAEs), which allow generation or reconstruction of observations by sampling from the learned latent representation. mcVAEs are described in more detail in Antelmi et al., “Sparse MultiChannel Variational Auto-encoder for the Joint Analysis of Heterogeneous Data”, Proceedings of the 36th International Conference on Machine Learning-PMLR, 2019, 302-311.

Given C data channels of different size and dimensions per observation, denoted X={x_{c}}_{c=1, . . . ,c}, a mcVAE estimates a joint latent space common to all channels. This latent space is usually represented by a lower-dimensional vector, denoted z, with multivariate Gaussian prior distribution, p (z). The joint log-likelihood for the observations, assuming each data channel to be conditionally independent from all others, can be expressed as:

$$ln p ( X \vert z , Θ ) = {\sum }_{i = 1}^{c} ln p ( {X}_{i} \vert z , {θ}_{i} )$$

where, θ={θ}_{i=1 . . . ,c} represents the set of parameters that define each channel's likelihood function. To discover the shared latent space z from which all data channels are assumed to be generated, the posterior distribution conditioned on the observations, i.e.p (z|x, θ) needs to be derived. As direct determination of this posterior distribution is intractable, variational inference may be used to compute an approximate posterior.

Compared to a VAE, a mcVAE is based on channel-specific encoding functions to map each channel in a joint latent space, and cross-channel decoding functions to simultaneously decode all the other channels from the latent representation. The variational posterior of the latent space of a mcVAE may be optimized to maximize data reconstruction. Since the latent representation into which a mcVAE encodes is shared, multiple channels may be decoded using the information encoded from a single channel. This feature allows one to attribute, impute or decode missing channels from the available ones. Hence, by first training the mcVAE with pairs of images (each pair comprising a captured fundus image 101 and a captured CMR image) from multiple training subjects, a decoded CMR image may be decoded, using the decoder 105, from the latent representation of a captured fundus image 101. The training of the mcVAE (i.e. the joint latent space 104 and the encoder 103 and decoder 105 architecture) is discussed in more detail below. The mcVAE may be sparse. The mcVAE may be sparse in the sense that each feature of the dataset (i.e., each dimension) depends on a small subset of latent factors. In more detail, dropout, a technique known in the art to regularize neural networks, can be naturally embedded in VAE to lead to a sparse representation of the variational parameters. Using a sparse mcVAE may ensure the evidence lower bound generally reaches the maximum value at convergence when the number of latent dimensions coincides with the true one used to generate the data. The evidence lower bound is a lower bound on the probability of observing some data under a model and can be used as an optimization criterion for approximating the posterior distribution p (z|x, θ) with a simpler (parametric) distribution.

A representation of the patient's heart 106 may be decoded 105 from the joint latent space 104. The format of the representation of the patient's heart 106 may depend on the format of the CMR images in the training data used in the training of the joint latent space 104. For example, the format of the representation of the patient's heart 106 may be the same as that of the CMR images used to train the joint latent space 104.

As depicted in FIG. 1, the representation of the patient's heart 106 may be provided as input to a neural network 107 configured to process the representation of the patient's heart 106. The neural network 107 configured to process the representation of the patient's heart 106 may be a convolutional neural network (CNN). The CNN may use a rectifier, such as ReLu, data augmentation techniques or regularization techniques such as “Dropout” (as described, for example, in Srinivastava et al., “Dropout: A Simple Way to Prevent Neural Networks from Overfitting”, Journal of Machine Learning Research 15 (2014) 1929-1958). The softmax layer of the CNN may be replaced with a fully connected regression layer with linear, sigmoid or other activation functions. For example, the CNN may be ResNet50 or VGG16. ResNet50 may be preferable to VGG16 due to the use of fewer parameters. More generally, the neural network 107 may comprise other attention-based networks, for example, self-attention or image transformers networks.

Instruments may provide a method of measuring patient characteristics. For example, a set of scales may provide a method of measuring the patient's weight. As depicted in FIG. 1, one or more patient characteristics 108, 113 may be received. Patient characteristics 108, 113 may be limited to such characteristics (for example, age and gender) that would be available to an optician, an ophthalmologist, or an eye clinic, thereby enabling the system to determine cardiac functional indices in less sophisticated clinical (or non-clinical) settings. Alternatively, wider patient characteristics 108, 113 may be used, for example, characteristics which would be available to a cardiologist or a cardiology department. More generally, the patient characteristics 108, 113 may comprise one or more of sex, age, gender, body mass index (BMI), weight, lean body mass, diastolic BP, systolic BP, HbA1c scores, blood glucose levels, cholesterol, smoking habits and alcohol consumption. Other additional patient characteristics may be used, such as data derived from the patient's history, blood samples, genetic profiling, microbiome analysis, etc.

As depicted in FIG. 1, one or more of the patient characteristics 108 are optionally processed by a neural network 109. The neural network 109 may be a fully connected neural network. Again, it will be appreciated that other appropriate network architectures may process the patient characteristics 108 in place of, or in addition to, the network 109 as will be known to the skilled person. The output of the neural network 109 may be used in further processing as described below.

As described above, cardiac functional indices 111 of the patient may be determined by a neural network 110 configured to estimate cardiac functional indices using the output from the neural network 107 configured to process the representation of the patient's heart 106 and, optionally (e.g. where present), the output from the neural network 109. For example, the neural network 110 configured to estimate cardiac functional indices may be referred to as a determination network. The determined cardiac functional indices 111 may be one or more of left ventricular mass (LVM) or left ventricular end-diastolic volume (LVEDV).

As described above, the determined cardiac functional indices 111 may be provided as input to a neural network 112 configured to predict the patient's risk of adverse cardiovascular events. Alternatively, the neural network 112 may be configured to use other techniques or architectures (other than CNNs) known to the skilled person. For example, the neural network 112 configured to predict the patient's risk of adverse cardiovascular events may be a CNN, particularly a CNN configured to calculate an output using logistic regression. Beneficially, the use of logistic regression eases interpretability, allowing comparisons between the coefficients of the variables used to predict the risk of adverse cardiovascular events.

Patient characteristics 113 may also be provided as an input to the neural network 112 configured to predict the patient's risk of adverse cardiovascular events. The patient characteristics 113 may comprise the characteristics discussed above in relation to the patient characteristics 108. The patient characteristics 113 may comprise different patient characteristics to (or may be a subset of) the patient characteristics 108 provided as an input to the neural network 109.

One or more of a patient's determined cardiac functional indices 111, the patient's predicted risk of adverse cardiovascular events 114, or another output from one of the neural networks 107, 110, 112 may be used in further activity. For example, outputs (of any stage) of the system 100 may be used to calculate premiums charged by a health insurance provider. A health insurance provider may accept a captured fundus image 101 and patient characteristics 108, 113 to be provided as part of a registration or renewal process. The health insurance provider may then use the method or system disclosed herein. Alternatively, the health insurance provider may accept the determined cardiac functional indices 111, predicted risk of adverse cardiovascular events 114, or another output from one of the neural networks 107, 110, 112 to be provided as part of a registration or renewal process. For example, a low predicted risk of the patient suffering an adverse cardiovascular event may indicate the patient is unlikely to need expensive medical care. So a health insurance provider may decrease the premium charged accordingly.

One or more of a patient's determined cardiac functional indices 111, the patient's predicted risk of adverse cardiovascular events 114 or another output from one of the neural networks 107, 110, 112 may be used to provide a notification or alert to the patient, another individual (e.g. a family member or carer) or a service provider. For example, upon the predicted risk of adverse cardiovascular events crossing a threshold, a patient (or the patient's clinician) may receive a notification informing them of the increase in the predicted risk.

One or more of a patient's determined cardiac functional indices 111, the patient's predicted risk of adverse cardiovascular events 114 or another output from one of the neural networks 107, 110, 112 may trigger the automatic generation and output of dietary and/or lifestyle adaptations. For example, based upon the predicted risk of adverse cardiovascular events crossing a threshold, a dietary or lifestyle adaptation may be generated and transmitted to the patient or the patient's clinician.

As will be understood to the skilled person, the system 100 may be modified. For example, the neural networks 107, 110 and 112 may be replaced by a single neural network for determining the risk of adverse cardiovascular events 114 from a representation of the patient's heart 106, the output from the neural network 109 and patient characteristics 113.

It will be appreciated that architecture of any of the neural networks 107, 109, 110 may be of diverse types, e.g., fully connected neural networks (multilayer perceptron), deep regression networks, transformer networks, etc. By way of example only, the neural network 109 may be a fully connected network and/or one or more layers in the neural network 109 may be replaced with one or more convolution layers. However, recent work has shown that other factors like data preprocessing dominate the performance, eclipsing nuances in the architecture, loss function, or activation functions [https://arxiv.org/abs/1803.08450].

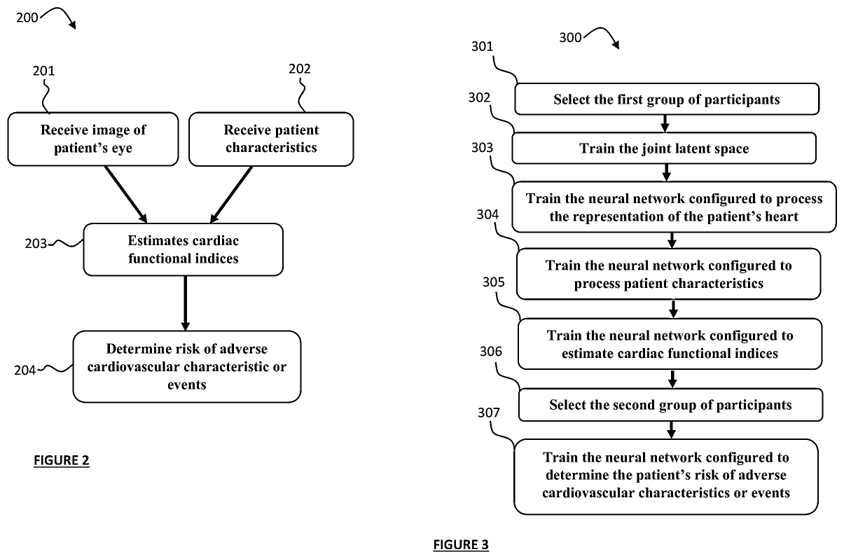

FIG. 2 depicts an example method 200 that may use the computer-implemented system 100 of FIG. 1 to determine the risk of adverse cardiovascular events. While method 200 determines a risk of adverse cardiovascular events, it will be appreciated that the method may end after outputting determined cardiac functional indices without determining a risk of cardiovascular events. At step 201, the computer-implemented system 100 receives a captured image of a patient's eye, such as the captured fundus image 101 of FIG. 1. At step 202, recorded patient characteristics, such as the patient characteristics 108, 113 of FIG. 1, are received. At step 203, the method estimates cardiac functional indices of the patient using the system 100 and as described above with respect to FIG. 1.

At step 204, following the determination of cardiac functional indices, the computer-implemented system may predict the patient's risk of adverse cardiovascular events 114 using the neural network 112 and as described above. As also described above, while FIG. 2 illustrates receipt of patient characteristics at step 202, it will be appreciated that the receipt and use of patient characteristics are not considered an essential feature of the method of FIG. 2.

FIG. 3 shows an example method 300 of training the system 100. The mcVAE (i.e. the encoder 103, joint latent space 104 and the decoder 105), the neural network 107 configured to process the representation of the patient's heart 106, neural network 109, the neural network 110 configured to estimate cardiac functional indices 111 and the neural network 112 configured to predict the patient's risk of adverse cardiovascular events are all trained. The method 300 is exemplary, and system 100 may be trained using other methods.

As the mcVAE provides the input for the neural network 107 configured to process the representation of the patient's heart, the mcVAE may be trained before training the neural network 107. As the neural network 107 configured to process the representation of the patient's heart 106 and the neural network 109 may provide the input for the neural network 110 configured to estimate cardiac functional indices, the neural network 107 and the neural network 109 may be trained before the training of the neural network 110. As the neural network configured 110 may provide the input for the neural network 112 configured to predict the patient's risk of adverse cardiovascular events, the neural network 110 may be trained before the training of the neural network 112.

At step 301, a first group of participants is selected from available training data. As described above, the training data comprises retinal images and CMR images. The retinal images and CMR images may take any appropriate form. By way of example only, in an example implementation, a suitable training dataset was obtained from the UK Biobank (UKB), referred to herein as the UKB dataset. The UKB dataset includes CMR images for participants who have undergone CMR imaging (for example using a clinical wide bore 1.5 Tesla MRI system (such as the MAG-NETOM Aera, Syngo Platform VD13A, Siemens Healthcare, Erlangen, Germany)) and retinal images for participants who have undergone retinal imaging using a Top-con 3D October 1000 Mark 2 (45° field-of-view, centred on and/or including both optic disc and macula). The UKB dataset contains data corresponding to 84,760 participants. The retinal images in the UKB dataset have an image resolution of 2048×1536 pixels (before any resampling).

The first group of participants (i.e. images of those participants) may be selected. The first group of participants may be selected by starting with a dataset (e.g. the full UKB dataset) and excluding a plurality of participants. Example criteria for excluding a plurality of participants are shown in Table 2, with further details on certain criteria below. Table 2 also shows the number of participants vetoed from the UKB dataset by each criterion when the criteria are applied in the order as shown in the Table. The first group of participants comprises 5,663 participants, following the criteria shown in Table 2. The first group of participants may be used to train the mcVAE, the neural network 107 configured to process the representation of the patient's heart, the neural network 109 and the neural network 110 configured to estimate cardiac functional indices.

TABLE 2
Example criteria for selecting the first group of participants

| | Number of participants vetoed |
|---|---|
| | from the UKB dataset when |
| | forming the first group |
| Criteria | of participants |
| A participant's data must include | 73,377 |
| a CMR image (and/or optionally | |
| patient characteristics) | |
| A participant's retinal image must | 5,044 |
| not be poor or very poor | |
| A participant must not have diabetes | 336 |
| A participant must not have had a | 293 |
| myocardial infarction previously | |
| A participant must not have | 14 |
| cardiomyopathy | |
| A participant must not undertake | 33 |
| strenuous exercise frequently | |

Participants may be excluded from the first group of participants due to a history of conditions known to affect LV mass. For example, participants may be excluded from the first group of participants due to a history of diabetes, previous myocardial infarction, cardiomyopathy or frequent strenuous exercise routines. Participants may be excluded from the first group of participants due to the participant data (for example, the captured fundus image) being of insufficient quality upon assessment of the data quality. For example, data quality may be determined by a deep learning method for quality assessment (QA) (as described, for example, in Fu et al. “Evaluation of Retinal Image Quality Assessment Networks in Different Color-spaces”, MICCAI 2019). Certain criteria may be specified prior to QA. Training and performance validation of the QA method may use an additional dataset, for example, EyePACS. EyePACS is a public dataset presented in the Kaggle platform for automatic diabetic retinopathy detection. Participants whose corresponding participant data fail the QA assessment may be excluded from the first group of participants. Participants not excluded may be identified as suitable for training and as having corresponding participant data comprising a good quality captured fundus image. Training may use all of the participants in the first group of participants or may use a proportion (for example, 25%, 50% or 75%) of the participants in the first group of participants.

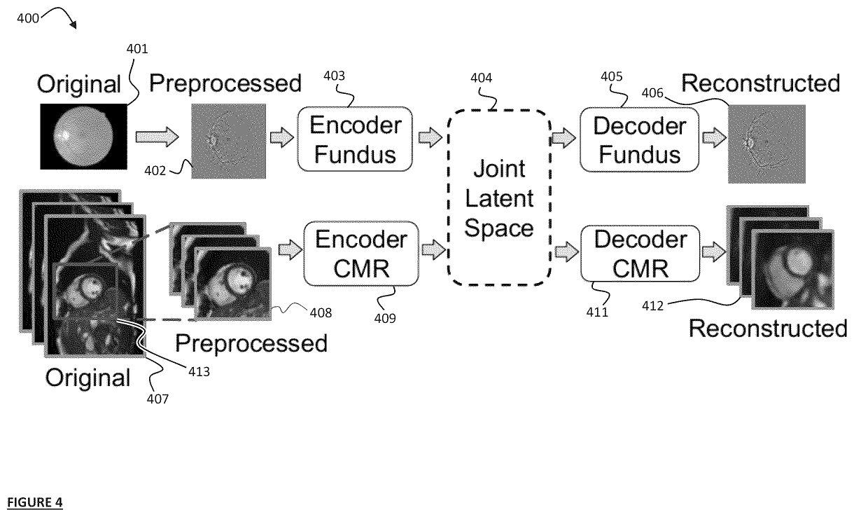

At step 302, a system 400 for training the mcVAE of FIG. 1 is trained as depicted in FIG. 4. After training of the system 400, an encoder 403 and a decoder 411 may be used as the encoder 103 and decoder 105 of FIG. 1.

Concerning FIG. 4, a captured fundus image 401 is received. Preprocessing may optionally be applied to produce a preprocessed fundus image 402. The preprocessing may comprise, for example, one or more of the preprocessing steps described above with respect to FIG. 1. Alternatively, the captured fundus image 401 may be suitable for use without preprocessing.

A captured CMR image 407 is also received. Preprocessing may, optionally, be applied to produce a preprocessed CMR image 408. Preprocessing may comprise, for example, detection of a region of interest (ROI) 413 around the heart depicted in the captured CMR image 407. Preprocessing may include cropping around the ROI 413. Advantageously, cropping may reduce the computational time and resources required to train the mc VAE, as the ROIs 413 are smaller, and the system 400 processes only the region of interest. Preprocessing of the captured CMR image 407 may comprise the use of deep learning techniques, for example, a CNN. For example, detection of the ROI 413 may comprise providing the captured CMR image 407 as input to a CNN. In one example, the CNN may be a U-Net or a variant of a U-Net (for example, as described in Ronneberger, Olaf; Fischer, Philipp; Brox, Thomas (2015). “U-Net: Convolutional Networks for Biomedical Image Segmentation”), to detect the heart in short-axis MRI stacks iteratively, from the basal slice to the apical slice.

As a further example of preprocessing that may be performed on the captured CMR image 407, each captured CMR image 407 may be resampled to a normalized volume. For example, each CMR image 407 may be resampled to a normalized volume, and the normalized volume may comprise the LV of the heart. In the example of training using data from the UKB dataset, each CMR image 407 may be resampled to 15 slices. Additionally, each captured CMR image 407 may be resampled to a particular resolution. For example, each captured CMR image 407 may be resampled to a 1 mm³ isotropic resolution. For example, each captured CMR image 407 may be resampled using cubic B-spline interpolation. Each captured CMR image 407 may be normalized, for example, by normalizing the range of pixel intensity values from 0 and 1.

The mcVAE may be trained using participant data, specifically from the first group of participants. In particular, two pairs of encoders 403, 409 and decoders 405, 411 (i.e. a first pair of an encoder 403 and a decoder 405 corresponding to a first data channel and a second pair of an encoder 409 and a decoder 411 corresponding to a second data channel) may be trained to encode to a joint latent space 404. It will be appreciated that after training, the joint latent space 404 may be the same as the joint latent space 104 of FIG. 1. The first data channel may comprise a fundus images data channel, and the second data channel may comprise a CMR image data channel or vice versa. The first pair, encoder 403 and decoder 405, may be trained on the first data channel (fundus images). The second pair of encoder 409 and a decoder 411 may be trained on the second data channel (CMR images). Example architectures for the encoders 403, 409 and the decoders 405, 411 are described in Table 1. The mcVAE may be trained using any suitable method, such as backpropagation, known to those skilled in the art. An example training strategy is described in Antelmi, Ayache, Robert and Lorenzi (Sparse Multi-Channel Variational Auto-encoder for the Joint Analysis of Heterogeneous Data (2019) Proc Int Conf Mach Learning, PMLR 97:302-311).

Returning to FIG. 3, at step 303 the neural network 107 conFigured to process the representation of the patient's heart 106 may be trained to output an abstract representation of the patient's heart 106. The neural network 107 may be trained using any suitable method, such as backpropagation. The neural network 107 may be trained on representations of participants' hearts. Training data for training the neural network 107 may be obtained from the first group of participants. The training data may use representations of the participants' hearts (decoded from the joint latent space representation). Alternatively, the representations of the participant's heart forming the training data for the mcVAE may be captured CMR images or preprocessed captured CMR images. Alternatively or additionally, training data may comprise delineations of the (captured or preprocessed captured) CMR images or delineations of the representations of the participants' hearts. Delineations may provide a measure of cardiac functional indices as a ground truth. Delineations may be automatic or manual. For example, manual delineations may be provided through the use of post-processing software (such as cvi42 from Circle Cardiovascular Imaging Inc.) and/or expert analysis. As an alternative, automatic delineations may be provided by methods known in the art (as described, for example, in Attar R, Pereañez M, Gooya A, Albá X, Zhang L, de Vila M H, Lee A M, Aung N, Lukaschuk E, Sanghvi M M, Fung K, Paiva J M, Piechnik S K, Neubauer S, Petersen S E, Frangi A F. Quantitative CMR population imaging on 20,000 subjects of the UK Biobank imaging study: LV/RV quantification pipeline and its evaluation. Med Image Anal. 2019 August; 56:26-42. DOI: 10.1016/j.media.2019.05.006).

At step 304, the neural network 109 may be trained to output an abstract representation of the patient characteristics 108. The neural network 109 may be trained using any suitable method, such as backpropagation. Training data for training the neural network 109 may comprise patient characteristics from the first group of participants. The patient characteristics may comprise the same characteristics discussed above concerning the patient characteristics 108, 113 in FIG. 1. Levels of Hb1A1c have been shown to positively correlate with cardiovascular mortality even in subjects without a history of diabetes. Therefore, the training data may comprise indications of HbA1c levels of participants, despite excluding participants from the first group of participants who have diabetes.

At step 305, the neural network 110 configured to output estimates of cardiac functional indices may be trained using any suitable method. For example, the neural network 110 may be trained using backpropagation. Training data for training the neural network 110 may comprise training data from the first group of participants. The neural network 110 may be trained separately from the neural networks 107 and 109. Alternatively, the neural network 110 may be trained together (i.e. end-to-end) with the neural networks 107, 109. In any event, the neural network 110 may be trained to output indications of cardiac functional indices in response to inputs comprising an output from the neural network 107 configured to process the representation of the patient's heart 106 (optionally together with an input comprising an output from the neural network 109). Ground truth for training the neural network 110 is therefore provided from the same data used to train the neural networks 107, 109 (such as the first group of participants). For example, taking a participant in the first group of participants having a fundus image and a CMR image and for whom cardiac functional indices are known, the neural network 110 may be trained to determine the correct cardiac functional indices from the output of the neural network 107 (where the neural network 107 receives as input a representation of the patient's heart generated by the decoder 411 in response to receipt by the encoder 409 of the fundus image of that participant).

At step 306, a second set of training data (e.g. a second group of participants from the group) may be selected. The second group of participants may be selected by excluding participants from a dataset (e.g. the UKB dataset). Example criteria for excluding participants are shown in Table 3. Table 3 also shows the number of participants excluded from the UKB dataset (in the example implementation) by each criterion when the criteria are applied in the order as shown in the Table. The second group of participants comprises 71,515 participants, following the criteria shown in Table 3. Other criteria may be considered by the skilled person to, for example, optimise the data available for training the neural network 112.

TABLE 3
Example criteria for selecting the second group of participants

| | Number of participants vetoed |
|---|---|
| | from the UKB dataset when |
| | forming the second |
| Criteria | group of participants |
| A participant's data must not include | 11,383 |
| a CMR image (and/or optionally patient | |
| characteristics) | |
| A participant must not have had a | 1,862 |
| myocardial infarction prior to the | |
| retinal image being taken | |

The second group of participants may comprise two classes, a first-class corresponding to participants who suffered an adverse cardiovascular event following the capture of the fundus image and a second class corresponding to participants who did not suffer an adverse cardiovascular event following the capture of the fundus image. The number of participants in each of the two classes may be unequal or non-similar; in other words, the data may be imbalanced. In response to imbalanced data, the data may be resampled wherein a subset of each resampled class of data is created to improve the time efficiency of training and prevent overtraining. For example, the majority class may be resampled and/or the minority class may be resampled. Beneficially, resampling of the majority class is a robust solution when the minority class comprises hundreds of cases, e.g. less than a thousand.

At step 307, the neural network 112 configured to predict the patient's risk of adverse cardiovascular events may be trained. Training data used to train the neural network 112 may comprise data from the second group of participants. The neural network 112 may be trained using cardiac functional indices (optionally together with patient characteristics) and known characteristics/incidents of adverse cardiac events. The cardiac functional indices may be the determined cardiac functional indices 111, or alternatively, measured cardiac functional indices. The performance of the neural network 112 in predicting a patient's risk of adverse cardiovascular events may be assessed using known statistical techniques. Cross-validation may be used to train and assess the neural network's performance 112. For example, K-fold cross-validation may be used, such as 10-fold cross-validation.

As mentioned above, cross-validation may be used to assess the performance of any component of the system 100. Following training, the performance of the system 100 may be assessed. Performance of the system 100 may be assessed using an additional dataset. For example, an alternative dataset (e.g. the AREDS database) may be used to provide validation plots.

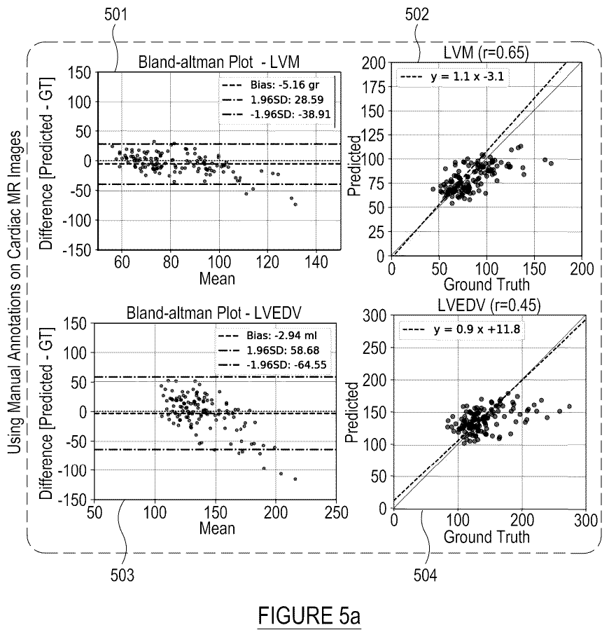

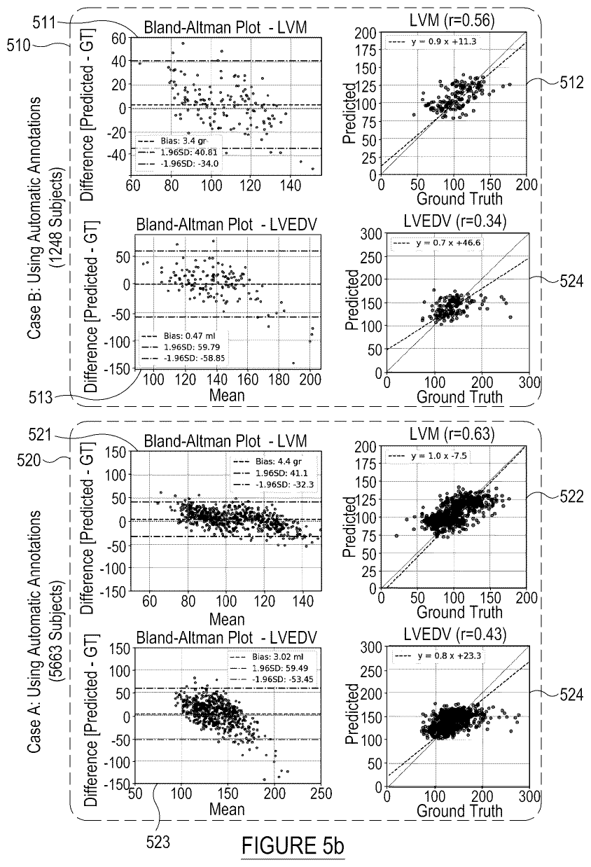

FIG. 5a shows a set of example Bland-Altman plots 501, 503 and correlation plots 502, 504 for cardiac functional indices determined using the system 100 compared to cardiac functional indices calculated using manual delineations (from a captured CMR image) using methods known in the art. The correlation coefficients (r) for LVM and LVEDV are 0.65 and 0.45, respectively. A subsample (approximately 10%) of the first group of participants was used to produce the plots 501, 502, 503, 504.

FIG. 5b shows a first set 510 and a second set 520 of example Bland-Altman plots 511, 513, 521, 521 and correlation plots 512, 514, 522, 524 for the determined cardiac functional indices (determined from the input captured fundus image) compared to the cardiac functional indices calculated from a captured CMR image. In the first set of plots 510, the calculated cardiac functional indices are determined manually by inspecting the captured CMR image by one or more clinicians. In the second set of plots 520, the calculated cardiac functional indices are calculated using automatic delineations using methods known in the art (for example, using the method described by Attar et al.). The correlation coefficients (r) for LVM and LVEDV are 0.56 and 0.34, respectively.

Table 2 compares the bias and limits of agreement of the determined cardiac functional indices 111 of the system 101 compared to prior art methods.

TABLE 2
A comparison of methods for estimating LVEDV and LVM. LoA relates to the 95% limits of agreement.

| | # of | | Cardiac | | |
|---|---|---|---|---|---|
| Reference | images | Method/Modality | index | Bias (LoA) | |
| Petersen et al | 50 | Inter-observer/CMR | LVEDV | −2.5 | (−7.5, 2.5) |
| | | | LVM | 2.5 | (−5, 8) |
| | | Intra-observer/CMR | LVEDV | −1 | (−5, 4) |
| | | | LVM | 1 | (−4, 5) |
| Bai et al. | 600 | Segmentation/CMR | LVEDV | 1 | (−20, 20) |
| | | | LVM | 1 | (−20, 20) |
| A [symbol] et al. | 4620 | Segmentation/CMR | LVEDV | −0.42 | (−26, 26) |
| | | | LVM | −2.03 | (−27, 22) |
| Siemens Sy [symbol] go Inline VF | 4674 | Segmentation/CMR | LVEDV | −2.5 | (−36.6, 3 [symbol] .5) |
| | | | LVM | 2 [symbol] .5 | (−5.4, 64.4) |
| Exp 1: This work/Manual Annotation | 1248 | Retinal & Demographic Data | LVEDV | −2.94 | (−64.55, 58.68) |
| | | | LVM | −5.16 | (−38.91, 28.59) |
| Exp 2A: This work/Automatic Annotation | 1248 | Retinal & Demographic Data | LVEDV | 0.47 | (−58.85, 59.79) |
| | | | LVM | 3.4 | (−34.00, 40.81) |
| Exp 2B: This work/Automatic Annotation | 5,663 | Retinal & Demographic Data | LVEDV | 3.02 | (−53.45, 59.49) |
| | | | LVM | 4.4 | (−32.30, 41.1) |

[symbol]

indicates data missing or illegible when filed

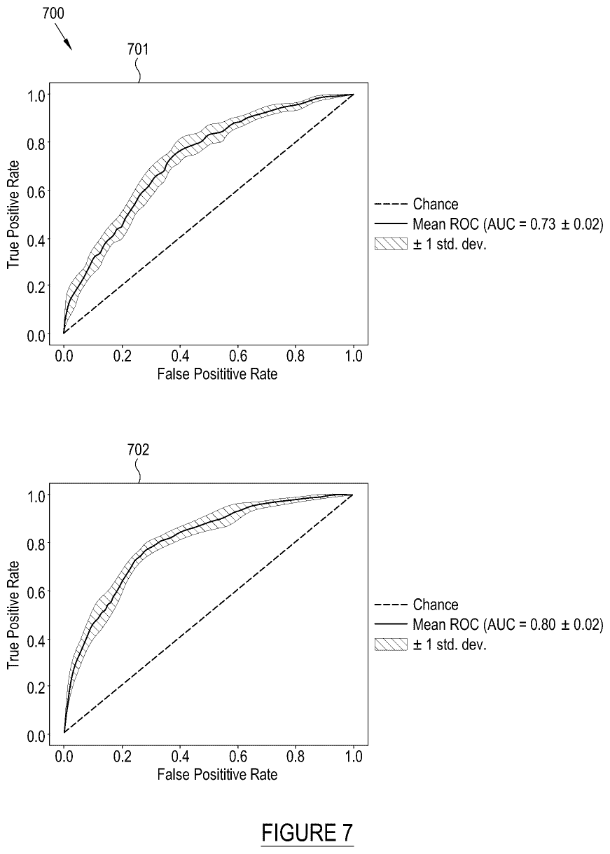

FIG. 6 shows three sets of plots 600. Each set of plots 610,620,630 shows the system's performance of FIG. 1 for estimating cardiac functional indices with different sizes of the captured fundus image (128×128 pixels, 256×256 pixels and 512×512 pixels, respectively). Each set of plots 610,620,630 comprises a Bland-Altman plot for the determined LVM 611,621,631, a Bland-Altman plot for the determined LVEDV 612,622,633, a correlation plot for the determined LVM 613,623,633, a correlation plot for the determined LVEDV 614,624,634 and a ROC curve for the predicted risk of adverse cardiovascular events 615, 625, 635. Each ROC curve 615, 625, 635 was calculated using the same method as shown in FIG. 7, discussed below.

FIG. 7 shows a set of example ROC curves 700 assessing the system's performance when predicting the risk of myocardial infarctions. Each ROC curve in the set of ROC curves 700 was produced using 10-fold cross-validation. The ROC curve 701 shows the performance of the system when using patient characteristics only, which has an accuracy of 0.66±0.03, a sensitivity of 0.7±0.04, a specificity of 0.64±0.03, precision of 0.64±0.03 and an F1 Score of 0.66±0.03. The ROC curve 702 shows the performance when using the determined cardiac functional indices and the demographic data, which has an accuracy of 0.74±0.03, a sensitivity of 0.76±0.02, a specificity of 0.73±0.05, a precision of 0.73±0.05, and an F1 score of 0.74±0.03. The ROC curve 702 shows an improvement compared to the ROC curve 701, demonstrating an improvement in the system's performance when the determined cardiac functional indices are used and the system's performance when the determined cardiac functional indices are used together with other patient characteristics.

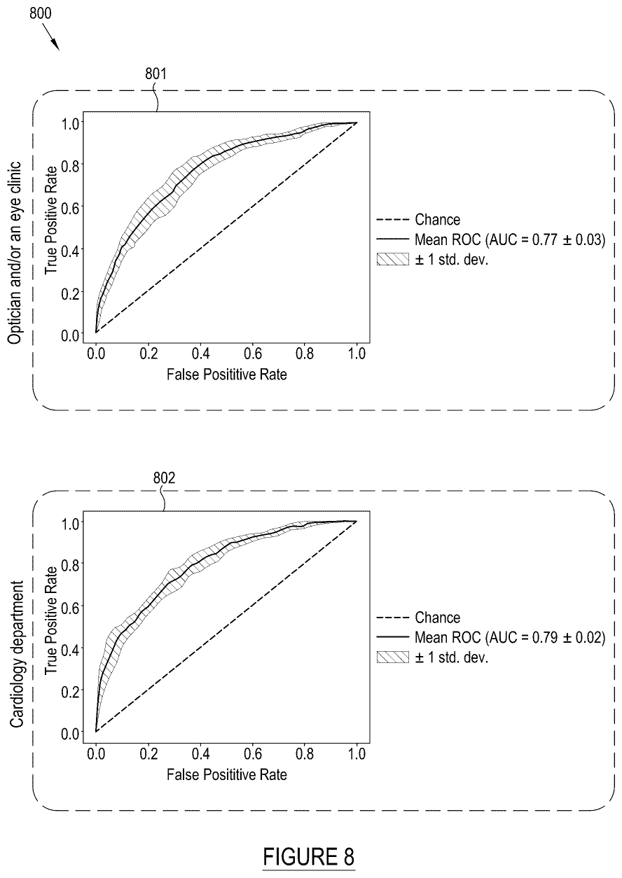

FIG. 8 shows a set of example ROC curves 800 assessing the performance of the system when predicting the risk of myocardial infarctions, produced following the same method as that used to produce the set of ROC curves 700 shown in FIG. 7. ROC curve 801 shows the performance of the system 100 when using a captured fundus image 101 and patient characteristics 108, 113 available at an optician or eye clinic. ROC curve 802 shows the performance of the system 100 when using a captured fundus image 101 and patient characteristics 108, 113 available at a cardiology department.

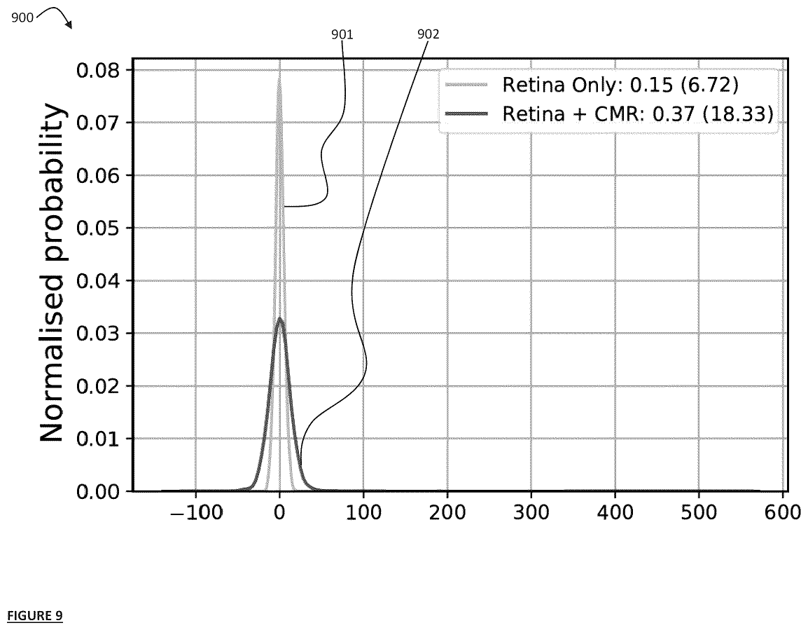

FIG. 9 shows plot 900, which shows example distributions of the latent variables encoded into the joint latent space 104. The first line 901 shows the distribution of the latent variables when only captured fundus images are used to train and are subsequently encoded into the joint latent space 104. The second line 902 shows the distribution of the latent variables when both captured fundus images and captured CMR images are used to train and are subsequently encoded into the joint latent space 104. The difference in the variance between the first line 901 and the second line 902 demonstrates that both captured retinal images and captured CMR images are encoded into the joint latent space 104. In other words, the variance is larger when (both) retinal, and CMR images are encoded (compared to when only retinal images are encoded) due to the latent variable encoding richer information coming from both the retinal and CMR images.

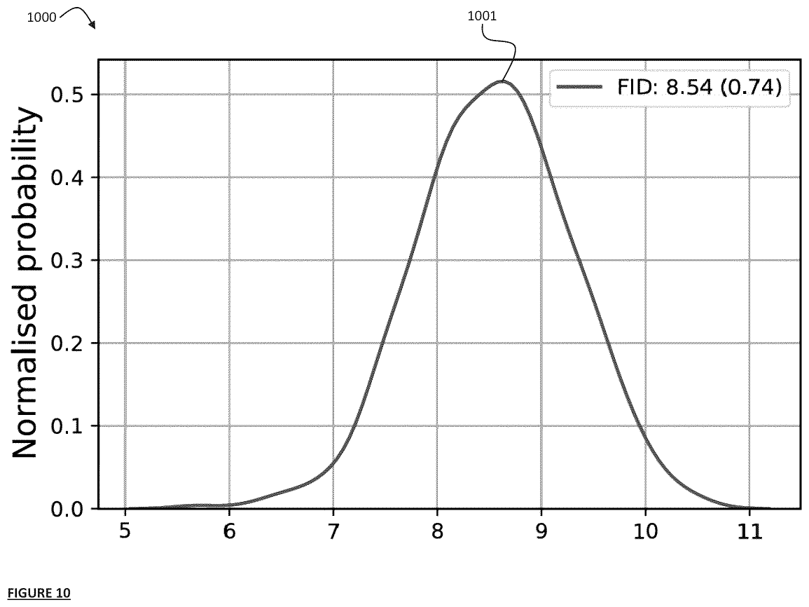

FIG. 10 show plot 1000, which shows an example distribution of the Frechet Inception Distance (FID) score 1001 calculated for the representations of the patients' hearts that are decoded from the joint latent space 104. The FID score may be used to capture the similarity of generated images to real images, as described, for example, by Heusel et al. (“GANs Trained by a Two Time-Scale Update Rule Converge to a Local Nash Equilibrium”, NIPS 2017). The FID score may be calculated using an ImageNet-pretrained Inception architecture. The distribution of the FID score 1001 shows that the representations of the patients' hearts are similar to the captured CMR images.

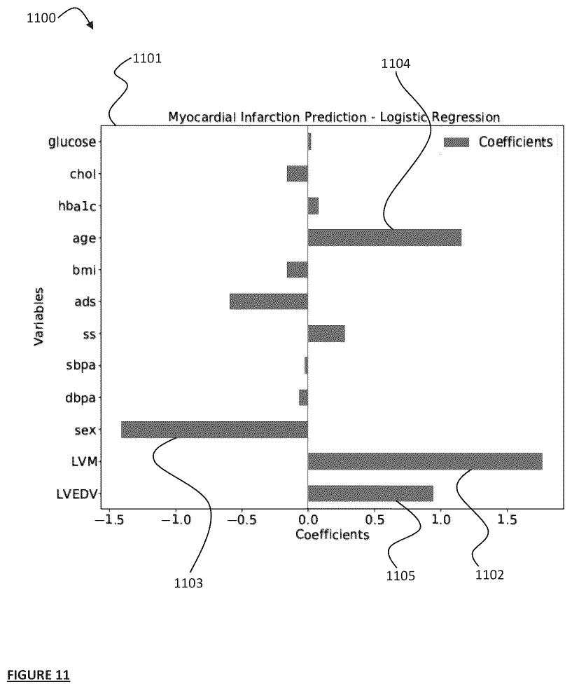

FIG. 11 shows an example set of values 1101 for the coefficients obtained from the neural network configured to predict the patient's risk of adverse cardiovascular events 112. The set of values 1101 shows that LVM 1102, sex 1103, age 1104 and LVEDV 1105 contribute most to predicting adverse cardiovascular events.

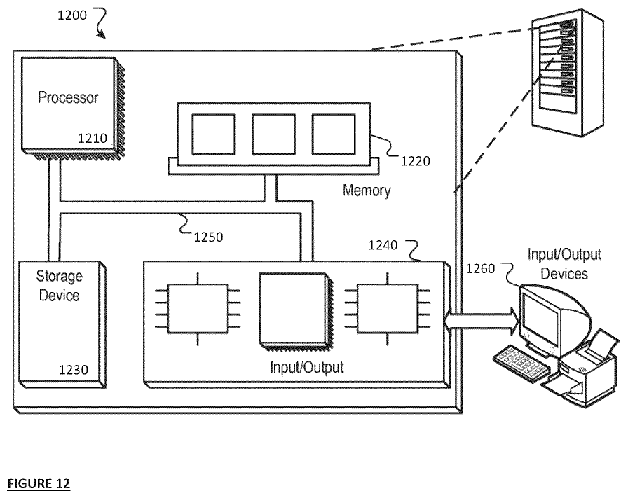

FIG. 12 shows an example computer system 1200. The computer-implemented system 100 may be implemented on a computer system, such as the computer system 1200. The computer system 1200 may comprise a central processing unit 1210, memory 1220, one or more storage devices 1230, an input/output processor 1240, circuitry to connect the components 1250 and one or more input/output devices 1260.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions, one or more programs include instructions that cause the apparatus to perform the operations or actions when executed by data processing.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively, or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example, a programmable processor, a computer, or multiple processors or computers. The apparatus can optionally include, in addition to hardware, code that creates an execution environment for computer programs, e.g., code that constitutes processor firmware, a protocol stack, a database management system, an operating system, or a combination of one or more of them. The apparatus can also be, or further include, special-purpose logic circuitry, e.g., an FPGA (field-programmable gate array) or an ASIC (application-specific integrated circuit).

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or another unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, subprograms, or portions of code. A computer program can be deployed to be executed on one computer or multiple computers located at one site or distributed across multiple sites and interconnected by a data communication network.

The processes and logic flow described in this specification can be performed by one or more programmable computers executing one or more computer programs to perform functions by operating on input data and generating output. The processes and logic flows can also be performed by special-purpose logic circuitry, e.g., an FPGA or an ASIC, or by a combination of special-purpose logic circuitry and one or more programmed computers.

Computers suitable for executing a computer program can be based on general or special-purpose microprocessors or both, or any other kind of central processing unit. The essential elements of a computer are a central processing unit for performing or executing instructions and one or more memory devices for storing instructions and data. Generally, a central processing unit will receive instructions and data from a read-only memory or a random access memory or both. The central processing unit and the memory can be supplemented by, or incorporated in, special-purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magnetooptical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that the user uses; for example, by sending web pages to a web browser on a user's device in response to requests received from the web browser. Also, a computer can interact with a user by sending text messages or other forms of a message to a personal device, e.g., a smartphone that is running a messaging application and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship between client and server arises by virtue of computer programs running on the respective computers and having a client-server relationship. In some embodiments, a server transmits data, e.g., an HTML page, to a user device, e.g., to display data to and receive user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially be claimed as such, one or more features from a claimed combination can in some cases be excised from the combination, and the claimed combination may be directed to a sub-combination or variation of a sub-combination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together into a single software product or packaged into multiple software products.

Particular embodiments of the subject matter have been described. Other embodiments are within the scope of the following claims. For example, the processes depicted in the accompanying figures do not necessarily require the particular order shown, or sequential order, to achieve desirable results. For example, the actions recited in the claims can be performed in a different order and still achieve desirable results. In some cases, multitasking and parallel processing may be advantageous.

1. A computer-implemented method for determining cardiac functional indices for a patient comprising at a first computing system comprising one or more processors:
receiving an image of a fundus of the patient;
encoding the received image into a joint latent space;
decoding from the joint latent space a representation of the patient's heart;
providing the representation decoded from the joint latent space to a neural network configured to generate cardiac functional indices; and
outputting the cardiac functional indices generated by the neural network in response to receiving the decoded representation of the patient's heart.

2. The method of claim 1, wherein the representation decoded from the joint latent space is an image of the patient's heart.

3. The method of claim 1, wherein the representation decoded from the joint latent space is an abstract representation of the patient's heart.

4. The method of claim 3, wherein the abstract representation is a lower-dimensional abstract representation than an image providing a visual representation of the patient's heart.

5. The method of claim 1, wherein the method further comprises processing a first characteristic of the patient at a neural network configured to process an input comprising the first characteristic and provide an output from the neural network configured to process the first characteristic as an input to the neural network configured to determine cardiac functional indices.

6. The method of claim 1, wherein the method further comprises use of a neural network configured to process the representation decoded from the joint latent space wherein an input to the neural network configured to process the representation decoded from the joint latent space comprises the representation decoded from the joint latent space and an output of the neural network configured to process the representation decoded from the joint latent space is provided as an input for the neural network configured to determine cardiac functional indices.

7. The method of claim 6, wherein the neural network configured to process the representation decoded from the joint latent space is a convolutional neural network (CNN).

8. The method of claim 1, wherein the determined cardiac functional indices comprise the left ventricular mass (LVM), the left ventricular end-diastolic volume (LVEDV), ejection fraction (EF), cardiac output (CO), LV end-systolic volume (LVESV), regional wall thickening (WT), regional wall motion (WM) and/or myocardial strains.

9. The method of claim 1, wherein the method further comprises determining the patient's risk of adverse cardiovascular characteristics or events.

10. The method of claim 1, wherein the method further comprises a neural network configured to determine the patient's risk of adverse cardiovascular characteristics/events wherein an input to the neural network for predicting the patient's risk of adverse cardiovascular characteristics/events comprises the determined cardiac functional indices.

11. The method of claim 10, wherein the input to the neural network for determining the patient's risk of adverse cardiovascular characteristics/events comprises second patient characteristics.

12. A non-transitory computer-readable media comprising instructions which, when executed by one or more computers, cause the one or more computers to determine cardiac functional indices for a patient comprising at the one or more computers:
receiving an image of a fundus of the patient;
encoding the received image into a joint latent space;
decoding from the joint latent space a representation of the patient's heart;
providing the representation decoded from the joint latent space to a neural network configured to generate cardiac functional indices; and
outputting the cardiac functional indices generated by the neural network in response to receiving the decoded representation of the patient's heart.

13. A computer system, comprising:
one or more processors,
one or more non-transitory computer readable media storing instructions configured to cause the one or more processors to-determine cardiac functional indices for a patient by:
receiving an image of a fundus of the patient;
encoding the received image into a joint latent space;
decoding from the joint latent space a representation of the patient's heart;
providing the representation decoded from the joint latent space to a neural network configured to generate cardiac functional indices; and
outputting the cardiac functional indices generated by the neural network in response to receiving the decoded representation of the patient's heart.

14. The computer system of claim 13 further comprising one or more wearable devices configured to capture the image of the fundus of the patient.
